(19) United States
(12) Patent Application Publication
(10) Pub. No.: US 2009/0012386 A1
(43) Pub. Date: Jan. 8, 2009

(54) SYSTEMS AND METHODS FOR PRE-OPERATIVELY IDENTIFYING FUNCTIONAL REGIONS OF A PATIENT'S BRAIN TO ASSIST IN THE PREPARATION OF A CONTEMPLATED SURGERY

(75) Inventor: Randy L. Buckner, Cambridge, MA (US)

Correspondence Address: CHOATE, HALL & STEWART LLP, TWO INTERNATIONAL PLACE, BOSTON, MA, 02110, US

(73) Assignee: THE PRESIDENT AND FELLOWS OF HARVARD COLLEGE, Cambridge, MA (US)

(21) Appl. No.: 12/166,942

(22) Filed: Jul. 2, 2008

Related U.S. Application Data
(60) Provisional application No. 60/948,342, filed on Jul. 6, 2007.

(51) Int. Cl. A61B 5/055

(52) U.S. Cl. 600/410

(57) ABSTRACT

Systems and methods for pre-operatively identifying functional regions of a patient's brain to assist in the preparation of a contemplated surgery. The systems and methods make use of correlations between regions of the patient's brain that exhibit spontaneous brain activity. The systems and methods may be used with any patient but solve a particular need for subjects that are unable to complete traditional task-based brain mapping studies.

### RELATED APPLICATIONS

The present application claims priority to U.S. Provisional Application Ser. No. 60/948,342, filed Jul. 6, 2007.

### BACKGROUND OF THE INVENTION

Many medical conditions, such as brain tumors and seizures, may exist for which surgically operating on a patient's brain is a viable treatment option. Such surgery may carry the risk of damaging functional areas of a patient's brain, which may leave a patient disabled following surgery. Existing methods of identifying functional areas of a patient's brain may require that a patient perform a task designed to elicit a given brain function while the patient undergoes an fMRI to monitor their brain activity. By identifying areas of the brain that are active during performance of the task, functional areas of the brain may be identified. However, this method may suffer from the drawback that it requires the patient to be conscious, alert, and compliant to instructions during the measurement. Thus there exists a need for a method of pre-operatively identifying functional areas of a patient's brain without requiring that a patient be able to perform a task on command.

### SUMMARY OF THE INVENTION

In one aspect, the present invention relates to a method for pre-operatively identifying functional regions of a patient's brain to assist in the preparation of a contemplated surgery using measurements of spontaneous brain activity. In some embodiments, the method includes: prior to a contemplated surgical operation on a patient's brain, taking at least one measurement of the patient's spontaneous brain activity levels at a plurality of regions in the brain; identifying, from the at least one measurement, a set of brain regions having an activity pattern correlated to each other; and producing an output indicating a location of the set of brain regions. For example, the output may include an image, an annotated image, or an indication that the regions are predominantly in a given brain hemisphere. The method may then include identifying the set of brain regions as regions to minimize disturbance of during the contemplated surgical operation. In some embodiments, a method may include determining a function performed by the set of brain regions and identifying, in response to determining the function the set of brain regions perform, the set of brain regions as regions to minimize disturbance of during the contemplated surgical operation. In some embodiments, the method may include determining, in response to the identification of the set of brain regions, to not perform the contemplated surgical operation. In other embodiments, the method may comprise determining the proximity of at least one of the set of brain regions to a potential site of the contemplated surgical operation. In one embodiment, this information may then be used to plan the surgery.

In another aspect, the present invention may relate to systems for pre-operatively identifying functional regions of a patient's brain to assist in the preparation of a contemplated surgery using measurements of spontaneous brain activity. In some embodiments, such systems may include MRI equipment. In some embodiments, such systems may include one or more databases of past brain activity measurements (which may be spontaneous and/or task driven) or previous functional area identifications. In some embodiments, such systems may include one or more computing devices for identifying correlations and mappings of measured spontaneous brain activity. In some embodiments, such systems may include one or more computing devices attached to a monitor, printer, or other data visualization apparatus.

### BRIEF DESCRIPTION OF THE DRAWINGS

The foregoing and other objects, aspects, features, and advantages of the invention will become more apparent and better understood by referring to the following description taken in conjunction with the accompanying drawings, in which:

FIG. 1 shows maps from two individual subjects constructed using analysis of spontaneous network correlations in rest scans;

FIG. 2 is a flow diagram of some embodiments of a method for pre-operatively identifying functional regions of a patient's brain using spontaneous brain activity; and

FIG. 3 is a block diagram of one embodiment of a system for pre-operatively identifying functional regions of a patient's brain using spontaneous brain activity.

### DETAILED DESCRIPTION OF THE INVENTION

Throughout this specification, any references referred to are hereby explicitly incorporated by such references. Research findings imply that a significant amount of brain activity persists all of the time, in the absence of immediate task goals as well as during tasks but independent of the task (See e.g. Buckner et al., 2007 for summary). Exploration of passive states (also referred to as resting or spontaneous states) is possible using modern imaging techniques (See e.g. Raichle et al., 2001). Analyses of the temporal dynamics of fMRI measured activity during rest have revealed that networks of regions spontaneously increase and decrease activity together in a correlated manner (Biswal et al., 1995; De Luca et al., 2002; Laufs et al., 2003; Greicius et al., 2003, 2004). Complex, structured activity patterns emerge in many brain systems (e.g., Biswal et al., 1995; Lowe et al., 1998; Hampson et al., 2002; Vincent et al., 2006, 2007; Fox et al., 2006). For example, a topographically distinct brain network supporting attention to the external environment also shows spontaneous correlations across its included regions, but with temporal dynamics that suggest that the externally directed network is independent or even opposed to the default network (Fransson, 2005, 2006; Fox et al., 2005). To give a specific example, five resting state networks that include distinct sensory, motor, and cognitive brain systems have been identified (De Luca et al. (2006).

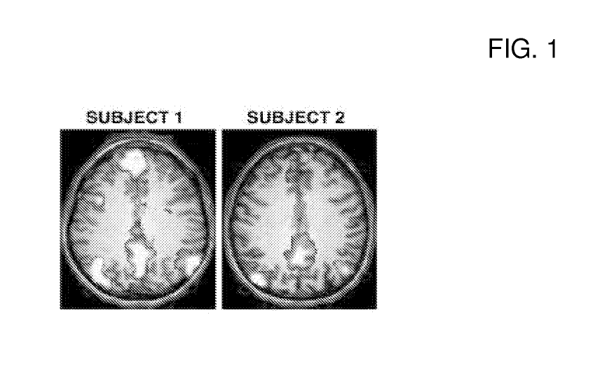

The spontaneous low-frequency network correlations identified in resting states are robust (e.g., Biswal et al., 1995; Greicius et al., 2003, 2004; Fransson, 2005, 2006; Fox et al., 2005; Vincent et al., 2006) and can be detected in individual subjects (Biswal et al., 1995; Vincent et al., 2006; Damoiseaux et al., 2006). As one illustration, FIG. 1 shows maps from two individual subjects constructed using analysis of spontaneous network correlations in rest scans. These data were acquired in about 20 min and show the regions spontaneously correlated with the hippocampal formation. Advantageously, rest activity measures may be more easily obtained than traditional task paradigms in patient groups because the task requirements to elicit them, can be minimal. As a result, the methods of the present invention provide a way to map functional brain regions in non-compliant subjects, such as might be useful for presurgical planning in cognitively impaired individuals (e.g., patients in a coma, children, elderly, patients with a cognitive deficiency, etc.). As but one example, it may be possible to determine the intrinsic lateralization of language pathways without having patients perform a language task. Structured spontaneous network correlations have even been shown to persist in patients under anesthesia (Vincent et al., 2007)

It is to be understood that spontaneous activity analysis may be amenable as a localization and/or a mapping tool. As but one example, rest activity correlations can be used with the hippocampal formation to define functional regions, in particular within parietal cortex and posterior midline regions, that are selectively responsive to memory processes (Vincent et al., 2006). This approach may be modified to survey the cortex broadly by using the complex topography of rest scan correlations to functionally segment the entire cortex (e.g., De Luca et al., 2006). Parcellations based on spontaneous activity patterns may be useful for defining multiple functional regions. In one embodiment, functional regions of a brain may be defined using a combination of spontaneous activity analysis and traditional task-based analysis (e.g., Tootell et al., 1995; Kanwisher et al., 1997).

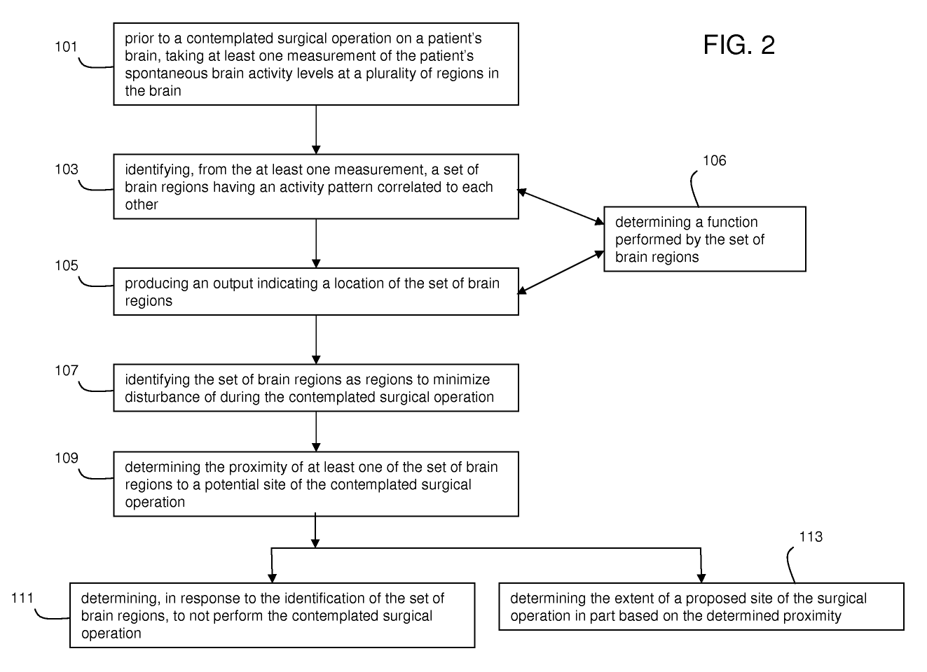

Referring now to FIG. 2, a method for pre-operatively identifying functional regions of a patient's brain to assist in the preparation of a contemplated surgery using measurements of spontaneous brain activity is shown. In brief overview, the method includes: prior to a contemplated surgical operation on a patient's brain, taking at least one measurement of the patient's spontaneous brain activity levels at a plurality of regions in the brain (step 101); identifying, from the at least one measurement, a set of brain regions having an activity pattern correlated to each other (step 103); and producing an output indicating a location of the set of brain regions (step 105). For example, the output may include an image, an annotated image, or an indication that the regions are predominantly in a given brain hemisphere. At any time before, during, or after these steps, the method may also include determining a function performed by the set of brain regions (step 106). The method may then include identifying the set of brain regions as regions to minimize disturbance of during the contemplated surgical operation (step 107). In some embodiments, the method may comprise determining the proximity of at least one of the set of brain regions to a potential site of the contemplated surgical operation (step 109). In some embodiments, the method may include determining, in response to the identification of the set of brain regions, to not perform the contemplated surgical operation (step 111). If the contemplated surgery is elected, the method may comprise determining the extent of a proposed site of the surgical operation in part based on the determined proximity (step 113). In one embodiment, the method may involve planning the surgery in light of the results of step 113.

Still referring to FIG. 2, now in greater detail, at least one measurement of the patient's spontaneous brain activity levels at a plurality of regions in the brain may be done in any manner (step 101). In some embodiments, the at least one measurement may comprise one or more MRI scans. An MRI scan may be any duration, including without limitation 1, 2, 5, 10, 15, 20, 25, 30, 40, 50, 60 or more minutes. In other embodiments, one of the at least one measurements may comprise any other method of monitoring brain activity levels in one or more brain regions.

The at least one measurement may be taken at any time or times prior to a contemplated surgical operation. In some embodiments, the measurements may be taken minutes, hours, days, weeks, or months before the contemplated surgical operation. A contemplated surgical operation may comprise any operation a patient or a doctor of the patient is considering performing on the patient. In some cases, a contemplated surgical operation may comprise an operation the patient or doctor has already decided will proceed. In other cases, a contemplated surgical operation may comprise an operation the patient or doctor has not yet decided will proceed. The surgical operation may comprise any surgical procedure involving the brain or head of the patient including, without limitation, tumor removals, resections and other surgical procedures to treat epilepsy, and, as technologies emerge, identification of functional brain areas for surgical implantation of stimulation devices and brain-computer interfaces

The at least one measurement may measure brain activity in any number of brain regions. A brain region may comprise any contiguous subset of a brain, and may be any size and shape, including without limitation a hemisphere, or lobe. In some embodiments, a brain region may comprise a discrete functional unit of the brain or a portion of a functional unit within the brain. The at least one measurement may be any resolution and may be directed at any portion of the brain. In some embodiments, the at least one measurement may measure brain activity over the entire brain.

The at least one measurement may be taken in any manner that allows spontaneous brain activity to be observed. In some embodiments, the measurements may be taken while a patient is resting or asleep. For example, a patient may be placed in an MRI machine and told to relax or attempt to sleep. In other embodiments, the measurements may be taken while a patient is sedated, comatose, unresponsive to instructions, or noncompliant to instructions. For example, a patient may be under anesthetics during some or all of the measurements. Or for example, a patient may be too young or old to understand or comply with verbal instructions. Or, for example, a patient may be suffering from one or more mental illnesses which impair the patient's ability to respond to instructions. In still other embodiments, the measurement may be performed while a patient is directed to perform one or more tasks. In these embodiments, the spontaneous brain activity may be differentiated from the directed brain activity using computational or other means. For example, a patient's brain activity during performance of a directed task may be compared with a patient's brain activity during a resting period in between tasks.

From the at least one measurement, a set of brain regions having an activity pattern correlated to each other may be identified in any manner (step 103). Any statistical, mathematical, visual, or other techniques may be used to identify or assist in identifying the correlation. In some embodiments, the correlation may comprise having elevated activity levels substantially simultaneously. In other embodiments, the correlation may comprise having elevated activity levels in sequence. In still other embodiments, the correlation may comprise having inversely related activity levels. For example, in some embodiments, a computer may analyze the output of the at least one measurement to identify regions which indicated relatively higher levels of activity substantially simultaneously. In other embodiments, a series of images may be produced comprising “snapshots” of the brain activity at given times. In this manner, regions of the brain having high activity at the same time may appear to be highlighted in the images.

Any type or form of output indicating a location of the set of brain regions may be produced (step 105). In some embodiments, the output may comprise an image which illustrates the location of the brain regions. In other embodiments, the output may comprise an indication whether the regions are predominantly in the left or right hemisphere of the brain. For example, the output may comprise an indication of whether the language regions of a patient's brain are predominantly on the left side of the brain. In still other embodiments, the output may indicate a statistical measure (e.g., a probability) that a given brain region is in a given location.

Determining a function performed by the set of brain regions may be optionally performed at any time and in any manner (step 106). In some embodiments, the function may be determined based on the locations of one or more of the brain regions corresponding to known locations of a given brain function. In other embodiments, the function may be determined based on a result of a brain measurement performed while the patient was directed to perform a given task. In some embodiments, a probability that a set of regions performs a given function may be determined.

The set of brain regions may be identified as regions to minimize disturbance of during the contemplated surgical operation in any manner (step 107). In some embodiments, the set of brain regions may be identified as such in response to a determination that the regions perform a given high-value function, such as language, sensory, cognition, or motor functions. In some embodiments, the regions may be identified as regions to completely avoid disturbance of. Disturbance of a region may comprise any procedure which will or may produce an effect on the region. For example, the set of brain regions may be identified as regions for which no surgery should be performed within a given distance of the regions. Or, for example, the set of brain regions may be identified as regions for which no surgery should be performed in the hemisphere of the brain in which they are predominantly located.

In some embodiments, the proximity of at least one of the set of brain regions to a potential site of the contemplated surgical operation may be determined (step 109). Proximity may be determined according to any measurement. In some embodiments, proximity may be measured by absolute distance. In other embodiments, proximity may be measured by location in the same hemisphere or lobe.

In some embodiments, the method may include determining, in response to the identification of the set of brain regions as regions to minimize disturbance of, to not perform the contemplated surgical operation (step 111). In addition to the identification of the brain regions, any other factors and information may be included in the decision to not perform the contemplated operation.

If the contemplated surgery is elected, the method may comprise determining the extent of a proposed site of the surgical operation in part based on the determined proximity (step 113). In some embodiment, the surgery may be planned in light of the results of step 113. For example, the proposed site of a surgical operation may be narrowed or moved in response to determining a high-value brain region is located near the surgical site.

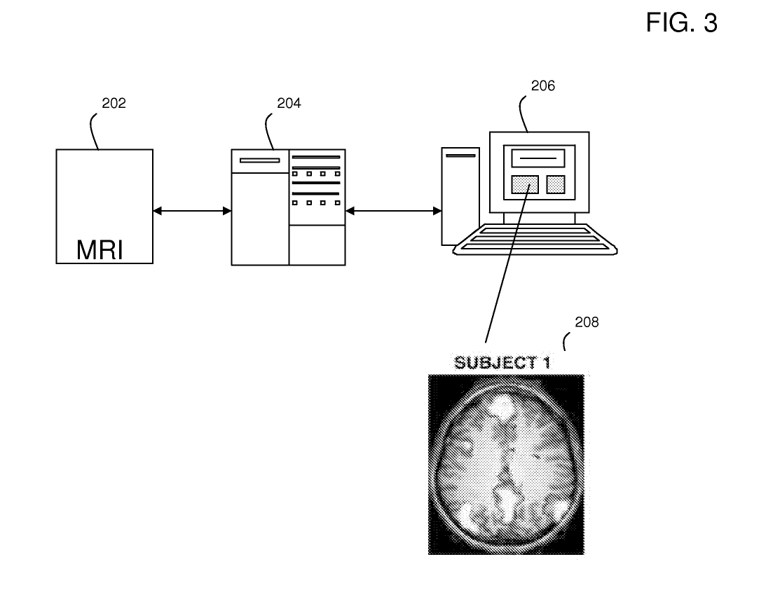

Referring now to FIG. 3, a block diagram of one embodiment of a system for pre-operatively identifying functional regions of a patient's brain using spontaneous brain activity is shown. The system comprises an MRI machine 202 which may perform the measurements of a patient's spontaneous brain activity at a time prior to a contemplated surgery. The results of the MRI may then be processed by one or more computing devices 204. The computing devices may process the MRI results in any manner, including identifying correlations, removing imperfections from the data set, producing images, identifying functional areas, and computing proximities of regions to brain sites. The system may then comprise an output viewing device 206. In the embodiment shown, the output comprises one or more images 208a, 208b which may identify functional areas of a patient's brain. The output may also comprise a textual readout, such as an indication of whether a region is located on the left or right hemisphere of a patient's brain. In other embodiments, the output may be printed.

Still referring to FIG. 2, a system for pre-operatively identifying functional regions of a patient's brain using spontaneous brain activity may include means for means for, prior to a contemplated surgical operation on a patient's brain, taking at least one measurement of the patient's spontaneous brain activity levels at a plurality of regions in the brain. Said means may include, without limitation, one or more MRI machines 202, or one or more positron emission tomography (PET) scanners.

The system may comprise any means for identifying, from the at least one measurement, a set of brain regions having an activity pattern correlated to each other. In some embodiments, the means for identifying the correlation may comprise a general-purpose computer executing software which analyzes the output of the at least one measurement. The software may comprise object code, assembly code, machine language, scripts, byte code, or any other type or form of instructions, and may identifying the correlation using any technique described herein. In other embodiments, the means for identifying the correlation may comprise special purpose hardware. In some embodiments, the means may comprise hardware and/or software contained within an MRI machine 202. In some embodiments, the means for identifying the correlated regions may be directly connected to the measurement device. In other embodiments, the means may be offsite. For example, the results of the at least one measurements may be sent in electronic or paper form to an offsite laboratory for identifying the correlated regions.

The system may comprise any means for producing an output indicating a location of the set of brain regions. In some embodiments, the means for producing output may comprise a display, including without limitation a CRT, LCD or projection display. The display may display an image or other output indicating the location of the regions. In other embodiments, the output means may comprise a printer, which may print images or textual output indication the locations of the regions.

The system may also comprise means for identifying the set of brain regions as regions to minimize disturbance of during the contemplated surgical operation. In some embodiments, these means may comprise software which identifies the specific function performed by the set of regions. In some embodiment, these means may comprise software which compares the location of the set of brain regions with the proposed surgical site (e.g., by overlaying images of the set of brain regions with anatomical images of the patient's brain). In certain embodiments, these means may comprise software for computing the distance or overlap between the set of brain regions and a proposed surgical site. In other embodiments, these means may comprise software for determining whether a majority of the regions are located on the same hemisphere as a proposed surgical site.

Software aspects of the present invention may be provided as one or more computer-readable programs embodied on or in one or more articles of manufacture. The article of manufacture may be a floppy disk, a hard disk, a CD-ROM, a flash memory card, a PROM, a RAM, a ROM, or a magnetic tape. In general, software programs may be implemented in any programming language, LISP, PERL, C, C++, PROLOG, or any byte code language such as JAVA. The software programs may be stored on or in one or more articles of manufacture as object code.

While the invention has been particularly shown and described with reference to specific preferred embodiments, it should be understood by those skilled in the art that various changes in form and detail may be made therein without departing from the spirit and scope of the invention as defined by the appended claims.

### REFERENCES

Biswal, B., Yetkin, F. Z., Haughton, V. M., Hyde, J. S., 1995. Functional connectivity in the motor cortex of resting human brain using echoplanar MRI. Magn. Reson. Med. 34, 537-541.

Buckner, R. L., Vincent, J. L., Unrest at rest: the importance of default activity and spontaneous network correlations, NeuroImage (2007) PMID: 17368915

Damoiseaux, J. S., Rombouts, S. A., Barkhof, F., Scheltens, P., Stam, C. J., Smith, S. M., Beckmann, C. F., 2006. Consistent resting-state networks across healthy subjects. Proc. Natl. Acad. Sci. U.S.A. 103, 13848-13853.

De Luca, M., Beckmann, C. F., Behrens, T., Clare, S., Mathews, P., De Stefano, N., Wollrich, M., Smith, S. M., 2002. Low frequency signals in fMRI—“resting state networks” and the “intensity normalization problem”. Proc. Int. Soc. of Magn. Reson. Med.

De Luca, M., Beckmann, C. F., De Stefano, N., Matthews, P. M., Smith, S. M., 2006. fMRI resting state networks define distinct modes of long-distance interactions in the human brain. NeuroImage 29, 1359-1367.

Fox, M. D., Corbetta, M., Snyder, A. Z., Vincent, J. L., Raichle, M. E., 2006. Spontaneous neuronal activity distinguishes human dorsal and ventral attention systems. Proc. Natl. Acad. Sci. U.S.A. 103, 10046-10051.

Fox, M. D., Snyder, A. Z., Vincent, J. L., Corbetta, M., Van Essen, D. C., Raichle, M. E., 2005. The human brain is intrinsically organized into dynamic, anticorrelated functional networks. Proc. Natl. Acad. Sci. U.S.A. 102, 9673-9678.

Fransson, P., 2005. Spontaneous low-frequency BOLD signal fluctuations: an fMRI investigation of the resting-state default mode of brain function hypothesis. Hum. Brain Mapp. 26, 15-29.

Fransson, P., 2006. How default is the default mode of brain function? Further evidence from intrinsic BOLD signal fluctuations. Neuropsychologia 44, 2836-2845.

Greicius, M. D., Krasnow, B., Reiss, A. L., Menon, V., 2003. Functional connectivity in the resting brain: a network analysis of the default mode hypothesis. Proc. Natl. Acad. Sci. U.S.A. 100, 253-258.

Greicius, M. D., Srivastava, G., Reiss, A. L., Menon, V., 2004. Default-mode network activity distinguishes Alzheimer's disease from healthy aging: evidence from functional MRI. Proc. Natl. Acad. Sci. U.S.A. 101, 4637-4642.

Hampson, M., Peterson, B. S., Skudlarski, P., Gatenby, J. C., Gore, J. C., 2002. Detection of functional connectivity using temporal correlations in MR images. Hum. Brain Mapp. 15, 247-262.

Kanwisher, N., McDermott, J., Chun, M. M., 1997. The fusiform face area: a module in human extrastriate cortex specialized for face perception. J. Neurosci. 17, 4302-4311.

Laufs, H., Krakow, K., Sterzer, P., Eger, E., Beyerle, A., Salek-Haddadi, A., Kleinschmidt, A., 2003. Electroencephalographic signatures of attention and cognitive default modes in spontaneous brain activity fluctuations at rest. Proc. Natl. Acad. Sci. U.S.A. 100, 11053-11058.

Lowe, M. J., Phillips, M. D., Lurito, J. T., Mattson, D., Dzemidzic, M., Mathews, V. P., 2002. Multiple sclerosis: low-frequency temporal blood oxygen level-dependent fluctuations indicate reduced functional connectivity initial results. Radiology 224, 184-192.

Raichle, M. E., MacLeod, A. M., Snyder, A. Z., Powers, W. J., Gusnard, D. A., Shulman, G. L., 2001. A default mode of brain function. Proc. Natl. Acad. Sci. U.S.A. 98, 676-682.

Tootell, R. B., Reppas, J. B., Kwong, K. K., Malach, R., Born, R. T., Brady, T. J., Rosen, B. R., Belliveau, J. W., 1995. Functional analysis of human MTand related visual cortical areas using magnetic resonance imaging. J. Neurosci. 15, 3215-3230.

Vincent, J. L., Patel, G. H., Fox, M. D., Snyder, A. Z., Baker, J. T., Van Essen, D. C., Zempel, J. M., Snyder, L. H., Corbetta, M., Raichle, M. E., 2007 in press. Intrinsic functional architecture in the anesthetized monkey brain. Nature.

Vincent, J. L., Snyder, A. Z., Fox, M. D., Shannon, B. J., Andrews, J. R., Raichle, M. E., Buckner, R. L., 2006. Coherent spontaneous activity identifies a hippocampal-parietal memory network. J. Neurophysiol. 96, 3517-3531.

We claim:

1. A method for pre-operatively identifying functional regions of a patient's brain to assist in the preparation of a contemplated surgery, the method comprising:
a. prior to a contemplated surgical operation on a patient's brain, taking at least one measurement of the patient's spontaneous brain activity levels at a plurality of regions in the brain;
b. identifying, from the at least one measurement, a set of brain regions having an activity pattern correlated to each other;
c. producing an output indicating a location of the set of brain regions; and
d. identifying the set of brain regions as regions to minimize disturbance of during the contemplated surgical operation.

2. The method of claim 1, wherein the at least one measurement is taken using functional magnetic resonance imaging.

3. The method of claim 1, wherein the patient is unconscious during at least a portion of the measurement.

4. The method of claim 1, wherein the patient is resting during at least a portion of the measurement.

5. The method of claim 1, wherein the patient is performing a task during at least a portion of the measurement, and wherein the spontaneous brain activity levels are measured independently of the task.

6. The method of claim 1, wherein the patient is anaesthetized during at least a portion of the measurement.

7. The method of claim 1, wherein the patient is non-compliant to instructions during at least a portion of the measurement.

8. The method of claim 1, wherein step (b) comprises identifying a set of brain regions demonstrating activity substantially simultaneously.

9. The method of claim 1, wherein step (c) comprises producing an image of the set of brain regions.

10. The method of claim 9, wherein step (d) comprises annotating the produced image.

11. The method of claim 1, wherein step (c) comprises producing an output indicating whether the set of brain regions are located predominantly in the right hemisphere, left hemisphere, or substantially evenly split among the left and right hemispheres.

12. The method of claim 1, wherein step (c) comprises producing an output indicating whether the language functions of the patient's brain are located predominantly in the right hemisphere, left hemisphere, or substantially evenly split among the left and right hemispheres.

13. The method of claim 1, wherein step (d) comprises determining the proximity of at least one of the set of brain regions to a potential site of the contemplated surgical operation.

14. The method of claim 13, further comprising determining the extent of a proposed site of the surgical operation in part based on the determined proximity.

15. The method of claim 13, wherein step (d) comprises producing an overlay of the set of brain regions and the proposed site of the contemplated surgical operation.

16. The method of claim 1, further comprising determining a function performed by the set of brain regions.

17. The method of claim 16, further comprising determining the set of brain regions performs one of speech, language, motor, or memory functions.

18. The method of claim 16, wherein step (d) comprises identifying, in response to determining the function the set of brain regions perform, the set of brain regions as regions to minimize disturbance of during the contemplated surgical operation.

19. The method of claim 1, further comprising taking a second measurement of the patient's brain activity levels while the patient is performing a directed task.

20. The method of claim 19, further comprising comparing the second measurement with the at least one measurement taken of the patient's spontaneous brain activity levels.

21. The method of claim 1, further comprising determining, in response to the identification of the set of brain regions, to not perform the contemplated surgical operation.

22. A system for pre-operatively identifying functional regions of a patient's brain to assist in the preparation of a contemplated surgery, the system comprising:
means for, prior to a contemplated surgical operation on a patient's brain, taking at least one measurement of the patient's spontaneous brain activity levels at a plurality of regions in the brain;
means for identifying, from the at least one measurement, a set of brain regions having an activity pattern correlated to each other;
means for producing an output indicating a location of the set of brain regions; and
means for identifying the set of brain regions as regions to minimize disturbance of during the contemplated surgical operation.

23. The system of claim 22, wherein the means for taking a measurement comprise an MRI machine.

24. The system of claim 22, wherein the means for producing an output indicating a location of the set of brain regions comprise one of: an LCD display, a CRT display, a projection display, or a printer.

25. The system of claim 22, wherein the means for identifying the set of brain regions as regions to minimize disturbance of during the contemplated surgical operation comprise computer readable program code comprising instructions for determining whether a majority of the set of regions are located in the same hemisphere as a proposed site of the contemplated surgical operation.

26. The system of claim 22, wherein the means for identifying the set of brain regions as regions to minimize disturbance of during the contemplated surgical operation comprise computer readable program code comprising instructions for determining the proximity of the set of regions to a proposed site of the contemplated surgical operation.
